(19) United States
(12) Patent Application Publication
(10) Pub. No.: US 2024/0354952 A1
(43) Pub. Date: Oct. 24, 2024

(54) SYSTEMS AND METHODS FOR BYPASS VESSEL RECONSTRUCTION

(71) Applicants: SHANGHAI UNITED IMAGING INTELLIGENCE CO., LTD., Shanghai (CN); UNITED IMAGING INTELLIGENCE (BEIJING) CO., LTD., Beijing (CN)

(72) Inventors: Zirong CHEN, Shanghai (CN); Dijia WU, Shanghai (CN); Pei DONG, Beijing (CN)

(73) Assignees: SHANGHAI UNITED IMAGING INTELLIGENCE CO., LTD., Shanghai (CN); UNITED IMAGING INTELLIGENCE (BEIJING) CO., LTD., Beijing (CN)

(21) Appl. No.: 18/759,821

(22) Filed: Jun. 29, 2024

Related U.S. Application Data
(63) Continuation of application No. PCT/CN2022/144130, filed on Dec. 30, 2022.

(30) Foreign Application Priority Data
Dec. 31, 2021 (CN) 202111669572.5
Dec. 31, 2021 (CN) 202111674821.X
Dec. 31, 2021 (CN) 202111674835.1

(51) Int. Cl. G06T 7/00; G06T 7/12; G06V 10/44; G06V 20/70; G16H 30/40

(52) U.S. Cl. CPC G06T 7/0012; G06T 7/12; G06V 10/44; G06V 20/70; G16H 30/40; G06T 2207/30048; G06T 2207/30101

(57) ABSTRACT

A system and a method for bypass vessel reconstruction may be provided. A target image including at least a cardiac region of a subject may be obtained. A first segmentation result and a second segmentation result may be determined based on the target image. The first segmentation result and the second segmentation result may respectively indicate the heart and vessels of the subject segmented from the target image. A target segment result may be determined using a vessel segment model based on the first segmentation result and the second segmentation result. The target segment result may include a plurality of segment labels of a plurality of points on the vessels of the subject, and the plurality of segment labels may include a segment label corresponding to bypass vessels. Further, data relating to one or more bypass vessels of the subject may be determined based on the target segment result.

### CROSS-REFERENCE TO RELATED APPLICATIONS

This application is a continuation of International Application No. PCT/CN2022/144130, filed on Dec. 30, 2022, which claims priority of Chinese Patent Applications No. 202111674835.1, 202111669572.5, and 202111674821.X, all filed on Dec. 31, 2021, the contents of each of which are incorporated herein by reference.

### TECHNICAL FIELD

The present disclosure relates to medical technology, and in particular, to systems and methods for bypass vessel reconstruction.

### BACKGROUND

A coronary artery bypass surgery (also referred to as a coronary artery bypass graft (CABG)) is a surgical procedure for coronary artery disease (CAD) aiming to relieve angina, stall progression of ischemic heart disease, and increase life expectancy. After the CABG of a patient is completed, a bypass vessel introduced by the CABG needs to be reconstructed based on a heart image of the patient, so that a user (e.g., a doctor) may evaluate a result of the CABG and formulate subsequent treatment plans according to the reconstruction result.

### SUMMARY

According to an aspect of the present disclosure, a system for bypass vessel reconstruction may be provided. The system may include at least one storage device including a set of instructions and at least one processor. The at least one processor may be configured to communicate with the at least one storage device. When executing the set of instructions, the at least one processor may be configured to direct the system to perform one or more of the following operations. The system may obtain a target image including at least a cardiac region of a subject. The system may determine a first segmentation result and a second segmentation result based on the target image. The first segmentation result may indicate the heart of the subject segmented from the target image, and the second segmentation result may indicate vessels of the subject segmented from the target image. The system may also determine a target segment result using a vessel segment model based on the first segmentation result and the second segmentation result. The target segment result may include a plurality of segment labels of a plurality of points on the vessels of the subject, and the plurality of segment labels may include a segment label corresponding to bypass vessels. The system may further determine data relating to one or more bypass vessels of the subject based on the target segment result.

In some embodiments, the vessels of the subject may include coronary arteries.

In some embodiments, to determine a first segmentation result and a second segmentation result based on the target image, the system may determine a first region of the cardiac region based on the first segmentation result from the target image. The system may further determine the second segmentation result based on the first region of cardiac region.

In some embodiments, to determine the second segmentation result based on first region of the cardiac region, the system may determine a second region of the cardiac region other than the first region from the target image. The system may further determine the second segmentation result by segmenting vessels from the first second region and the second region.

In some embodiments, to determine a second region of the cardiac region other than the first region from the target image, the system may generate a first vessel segmentation result by segmenting vessels from the first region. The system may also determine an initial segment result using the vessel segment model based on the first segmentation result and the first vessel segmentation result. The system may further determine whether there are one or more bypass vessels in the subject based on the initial segment result. In response to determining that there are one or more bypass vessels in the subject, the system may determine the second region from the target image.

In some embodiments, the vessel segment model may include a normal vessel segment model and a bypass vessel segment model. The bypass vessel segment model may be trained using first training samples, the normal vessel segment model may be trained using second training samples, and a proportion of sample data relating to bypass vessels in the first training samples may be greater than that in the second training sample. The initial segment result may be determined using the normal vessel segment model. The target segment result may be determined using the bypass vessel segment model.

In some embodiments, to determine whether there are one or more bypass vessels in the subject based on the initial segment result, the system may determine a plurality of vessel connected components based on the first vessel segmentation result. For each of the plurality of vessel connected components, the system may determine feature information of the vessel connected component. The feature information of the vessel connected component may include at least one of a distance from the vessel connected component to a ventricle region, a distance from the vessel connected component to an aorta region, or a size of the vessel connected component. The system may further determine whether there are one or more bypass vessels in the subject based on the feature information relating to the plurality of vessel connected components.

In some embodiments, to determine a target segment result using a vessel segment model based on the first segmentation result and the second segmentation result, the system may determine a first distance map including distance information from each point to a ventricle region in the first segmentation result based on the first segmentation result. The system may also determine a second distance map including distance information from each point to an aorta region in the first segmentation result based on the first segmentation result. The system may further determine the target segment result using the vessel segment model based on the first segmentation result, the second segmentation result, the first distance map, and the second distance map.

In some embodiments, the data relating to the one or more bypass vessels may include at least one of first data relating to a starting point of each bypass vessel, second data relating to a path of each bypass vessel, or third data relating to an anastomotic stoma between each bypass vessel and coronary arteries.

In some embodiments, the first data relating to the starting point of each bypass vessel may be determined by performing the following operations. The system may determine endpoints of centerlines of the plurality of vessels based on the target segment result. The system may also determine one or more candidate starting points based on the target segment result from the endpoints of the centerlines of the vessels. The system may further determine the starting point of each bypass vessel based on the first segmentation result from the one or more candidate starting points.

In some embodiments, to determine data relating to one or more bypass vessels based on the target segment result, the system may determine the first data relating to the starting point of each bypass vessel based on the target segment result. The system may also determine graph data corresponding to the starting point of each bypass vessel. The graph data may be represented by a centerline tree that is generated based on points on one or more centerlines of the vessels and has the starting point as a root node. The system may further determine at least one of the second data or the third data based on the graph data corresponding to the starting point of each bypass vessel.

In some embodiments, the centerline tree may include nodes and edges. To determine at least one of the second data or the third data based on the graph data corresponding to the starting point of each bypass vessel, the system may determine segment labels corresponding to the edges and the nodes of the centerline tree based on the target segment result. The system may further determine the second data relating to the path of each bypass vessel based on the segment labels corresponding to the edges of the centerline tree and the nodes of the centerline tree.

In some embodiments, to determine at least one of the second data or the third data based on the graph data corresponding to the starting point of each bypass vessel, for each of the one or more bypass vessel, the system may perform one or more of the following operations. The system may determine a termination point of the path of the bypass vessel and the segment label corresponding to the termination point of the path of the bypass vessel based on the second data relating to the path of the bypass vessel. The system may further determine the third data relating to the anastomotic stoma between the bypass vessel and other vessels by tracing the points on the path of the bypass vessel from the termination point towards the starting point of the bypass vessel based on the segment label corresponding to the termination point of the path of the each bypass vessel.

According to another aspect of the present disclosure, a method for bypass vessel reconstruction may be provided. The method may include obtaining a target image including at least a cardiac region of a subject. The method may include determining a first segmentation result and a second segmentation result based on the target image. The first segmentation result may indicate the heart of the subject segmented from the target image, and the second segmentation result may indicate vessels of the subject segmented from the target image. The method may also include determining a target segment result using a vessel segment model based on the first segmentation result and the second segmentation result. The target segment result may include a plurality of segment labels of a plurality of points on the vessels of the subject, and the plurality of segment labels may include a segment label corresponding to bypass vessels. The method may further include determining data relating to one or more bypass vessels of the subject based on the target segment result.

According to yet another aspect of the present disclosure, a system for bypass vessel reconstruction may be provided. The system may include an acquisition module and a determination module. The acquisition module may be configured to obtain a target image including at least a cardiac region of a subject. The determination module may be configured to determine a first segmentation result and a second segmentation result based on the target image. The first segmentation result may indicate the heart of the subject segmented from the target image, and the second segmentation result may indicate vessels of the subject segmented from the target image. The determination module may be also configured to determine a target segment result using a vessel segment model based on the first segmentation result and the second segmentation result. The target segment result may include a plurality of segment labels of a plurality of points on the vessels of the subject, and the plurality of segment labels may include a segment label corresponding to bypass vessels. The determination module may be further configured to determine data relating to one or more bypass vessels of the subject based on the target segment result.

According to yet another aspect of the present disclosure, a non-transitory computer readable medium may be provided. The non-transitory computer readable medium may include at least one set of instructions for bypass vessel reconstruction. When executed by one or more processors of a computing device, the at least one set of instructions may cause the computing device to perform a method. The method may include obtaining a target image including at least a cardiac region of a subject. The method may include determining a first segmentation result and a second segmentation result based on the target image. The first segmentation result may indicate the heart of the subject segmented from the target image, and the second segmentation result may indicate vessels of the subject segmented from the target image. The method may also include determining a target segment result using a vessel segment model based on the first segmentation result and the second segmentation result. The target segment result may include a plurality of segment labels of a plurality of points on the vessels of the subject, and the plurality of segment labels may include a segment label corresponding to bypass vessels. The method may further include determining data relating to one or more bypass vessels of the subject based on the target segment result.

According to yet another aspect of the present disclosure, a device for bypass vessel reconstruction may be provided. The device may include at least one processor and at least one storage device for storing a set of instructions. When the set of instructions may be executed by the at least one processor, the device performs the method for bypass vessel reconstruction.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a block diagram illustrating exemplary processing device according to some embodiments of the present disclosure;

FIG. 3 is a flowchart illustrating an exemplary process for determining data relating to one or more bypass vessels of a subject according to some embodiments of the present disclosure;

FIG. 4 is a flowchart illustrating an exemplary process for determining a second segmentation result according to some embodiments of the present disclosure;

FIG. 5 is a schematic diagram illustrating an exemplary process for reconstructing one or more bypass vessels of a subject according to some embodiments of the present disclosure;

FIG. 6 is a flowchart illustrating an exemplary process for determining data relating to one or more bypass vessels according to some embodiments of the present disclosure; and

FIG. 7 is a schematic diagram illustrating an exemplary centerline tree according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “pixel” and “voxel” in the present disclosure are used interchangeably to refer to an element of an image. An anatomical structure shown in an image of a subject (e.g., a patient) may correspond to an actual anatomical structure existing in or on the subject's body.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, a representation of a subject (e.g., an object, a patient, or a portion thereof) in an image may be referred to as “subject” for brevity. For instance, a representation of an organ, tissue (e.g., a heart, a liver, a lung), or an ROI in an image may be referred to as the organ, tissue, or ROI, for brevity. Further, an image including a representation of a subject, or a portion thereof, may be referred to as an image of the subject, or a portion thereof, or an image including the subject, or a portion thereof, for brevity. Still further, an operation performed on a representation of a subject, or a portion thereof, in an image may be referred to as an operation performed on the subject, or a portion thereof, for brevity. For instance, a segmentation of a portion of an image including a representation of an ROI from the image may be referred to as a segmentation of the ROI for brevity.

The present disclosure primarily relates to bypass vessel reconstruction. As used herein, bypass vessel reconstruction may involve determining data relating to one or more bypass vessels of a subject and/or generating a reconstruction image or model of the bypass vessel(s).

Conventionally, after a CABG of a patient is completed, a user (e.g., a doctor) needs to manually reconstruct a bypass vessel introduced by the CABG. Specifically, coronary arteries are segmented from an image including a cardiac region of a patient and divided into vessel segments by an existing coronary segmentation and segment system. Since the existing coronary segmentation and segment system is unable to divide the bypass vessel into vessel segment automatically, the user needs to manually determine data relating to the bypass vessel (e.g., a starting point, a path, an anastomotic stoma between the bypass vessel and other vessels) based on the segment result of the coronary arteries for reconstructing an image or a model of the bypass vessel. However, compared with the coronary arteries, the bypass vessel is normally longer and has a more complex trajectory and a poorer imaging visualization. The conventional approach for determining the data relating to the bypass vessel may be inefficient and/or susceptible to human errors or subjectivity. Thus, it may be desirable to develop systems and methods for automatically determining the data relating to the bypass vessel, thereby improving the efficiency and/or accuracy of bypass vessel reconstruction. The terms “automatic” and “automated” are used interchangeably referring to methods and systems that analyze information and generates results with little or no direct human intervention.

An aspect of the present disclosure relates to systems and methods for determining data relating to one or more bypass vessels. The systems may obtain a target image including at least a cardiac region of a subject. The systems may determine a first segmentation result and a second segmentation result based on the target image. The first segmentation result may indicate the heart of the subject segmented from the target image, and the second segmentation result may indicate vessels of the subject segmented from the target image. The systems may also automatically and accurately determine a target segment result using a vessel segment model based on the first segmentation result and the second segmentation result. The target segment result may include a plurality of segment labels of a plurality of points on the vessels of the subject, and the plurality of segment labels include a segment label corresponding to bypass vessels. The systems may further determine data relating to one or more bypass vessels of the subject based on the target segment result. In some embodiments, the data relating to the one or more bypass vessels may include first data relating to a starting point of each bypass vessel, second data relating to a path of each bypass vessel, third data relating to an anastomotic stoma between each bypass vessel and other vessels, or the like, or any combination thereof. Compared with the conventional approach, the systems and methods of the present disclosure is more efficient and accurate by, e.g., reducing the workload of a user, cross-user variations, and the time needed for determining the data relating to the bypass vessel, thereby improving the accuracy and efficiency of the reconstruction of the bypass vessel(s).

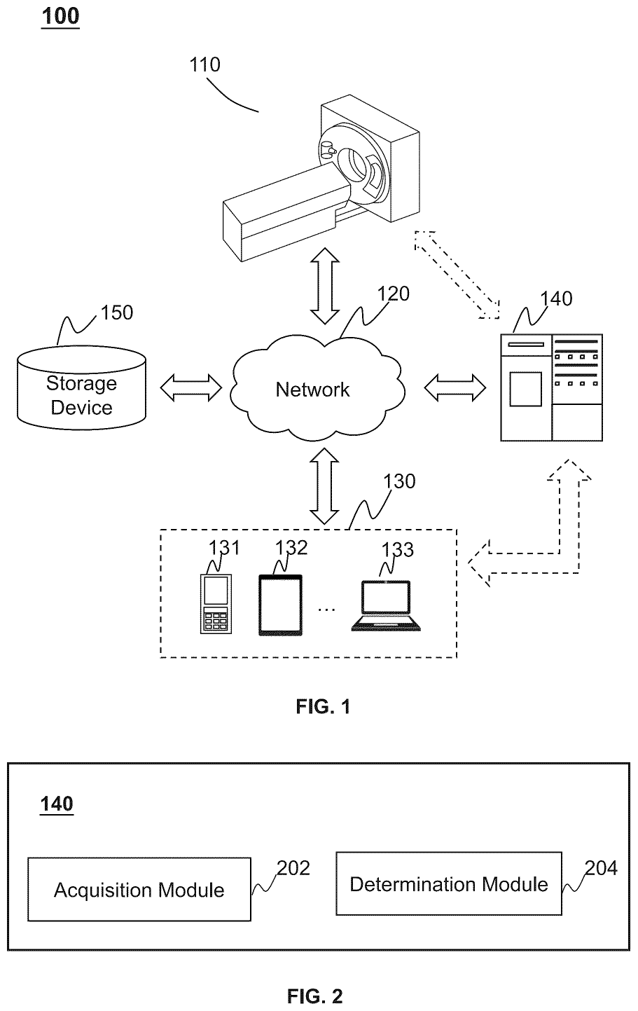

FIG. 1 is a schematic diagram illustrating an exemplary imaging system 100 according to some embodiments of the present disclosure. As shown in FIG. 1, the imaging system 100 may include an imaging device 110, a network 120, one or more terminals 130, a processing device 140, and a storage device 150. In some embodiments, the imaging device 110, the terminal(s) 130, the processing device 140, and/or the storage device 150 may be connected to and/or communicate with each other via a wireless connection (e.g., the network 120), a wired connection, or a combination thereof. The connection between the components of the imaging system 100 may be variable.

The imaging device 110 may be configured to scan a subject (or a part of the subject) to acquire medical image data associated with the subject. The medial image data relating to the subject may be used for generating an anatomical image (e.g., a CTA image, an MRI image) of the subject. The anatomical image may illustrate an internal structure of the subject. In some embodiments, the imaging device 110 may include a single-modality scanner and/or multi-modality scanner. The single modality scanner may include, for example, an X-ray scanner, a CT scanner, a magnetic resonance imaging (MRI) scanner, an ultrasonography scanner, a positron emission tomography (PET) scanner, a Digital Radiography (DR) scanner, or the like, or any combination thereof. The multi-modality scanner may include, for example, an X-ray imaging-magnetic resonance imaging (X-ray-MRI) scanner, a positron emission tomography-X-ray imaging (PET-X-ray) scanner, a single-photon emission computed tomography-magnetic resonance imaging (SPECT-MRI) scanner, a positron emission tomography-computed tomography (PET-CT) scanner, etc. It should be noted that the imaging device 110 described below is merely provided for illustration purposes, and not intended to limit the scope of the present disclosure.

The network 120 may include any suitable network that can facilitate the exchange of information and/or data for the imaging system 100. In some embodiments, one or more components of the imaging system 100 (e.g., the imaging device 110, the processing device 140, the storage device 150, the terminal(s) 130) may communicate information and/or data with one or more other components of the imaging system 100 via the network 120. For example, the processing device 140 may obtain image data from the imaging device 110 via the network 120.

The terminal(s) 130 may be connected to and/or communicate with the imaging device 110, the processing device 140, and/or the storage device 150. For example, the terminal(s) 130 may receive a user instruction to determine data relating to one or more bypass vessels of the subject. As another example, the terminal(s) 130 may display a reconstructed image or model of the bypass vessel(s) of the subject. In some embodiments, the terminal(s) 130 may include a mobile device 131, a tablet computer 132, a laptop computer 133, or the like, or any combination thereof. In some embodiments, the terminal(s) 130 may be part of the processing device 140.

The processing device 140 may process data and/or information obtained from the imaging device 110, the storage device 150, the terminal(s) 130, or other components of the imaging system 100. For example, the processing device 140 may obtain a target image including at least a cardiac region of a subject from the imaging device 110 or the storage device 150. Further, the processing device 140 may determine data relating to one or more bypass vessels of the subject based on the target image. As another example, the processing device 140 may generate one or more machine learning models (e.g., a first segmentation model, a second segmentation model, a vessel segment model) that can be used to determine the data relating to the bypass vessel(s) of the subject.

In some embodiments, the processing device 140 (e.g., one or more modules illustrated in FIG. 2) may execute instructions and may accordingly be directed to perform one or more processes (e.g., process 300, 400, and 600) described in the present disclosure. For example, each of the one or more processes may be stored in a storage device (e.g., the storage device 150) as a form of instructions, and invoked and/or executed by the processing device 140.

In some embodiments, the processing device 140 may be a single server or a server group. In some embodiments, the processing device 140 may be local to or remote from the imaging system 100. Merely for illustration, only one processing device 140 is described in the imaging system 100. However, it should be noted that the imaging system 100 in the present disclosure may also include multiple processing devices. Thus operations and/or method steps that are performed by one processing device 140 as described in the present disclosure may also be jointly or separately performed by the multiple processing devices. For example, if in the present disclosure the processing device 140 of the imaging system 100 executes both process A and process B, it should be understood that the process A and the process B may also be performed by two or more different processing devices jointly or separately in the imaging system 100 (e.g., a first processing device executes process A and a second processing device executes process B, or the first and second processing devices jointly execute processes A and B).

The storage device 150 may store data, instructions, and/or any other information. In some embodiments, the storage device 150 may store data obtained from the processing device 140, the terminal(s) 130, and/or the imaging device 110. For example, the storage device 150 may store image data collected by the imaging device 110. As another example, the storage device 130 may store the data relating to one or more bypass vessels of the subject. In some embodiments, the storage device 150 may store data and/or instructions that the processing device 140 may execute or use to perform exemplary methods described in the present disclosure.

It should be noted that the above description of the imaging system 100 is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the imaging system 100 may include one or more additional components. Additionally or alternatively, one or more components of the imaging system 100 described above may be omitted. As another example, two or more components of the imaging system 100 may be integrated into a single component.

FIG. 2 is a block diagram illustrating exemplary processing device 140 according to some embodiments of the present disclosure.

As shown in FIG. 2, the processing device 140 may include an acquisition module 202 and a determination module 204.

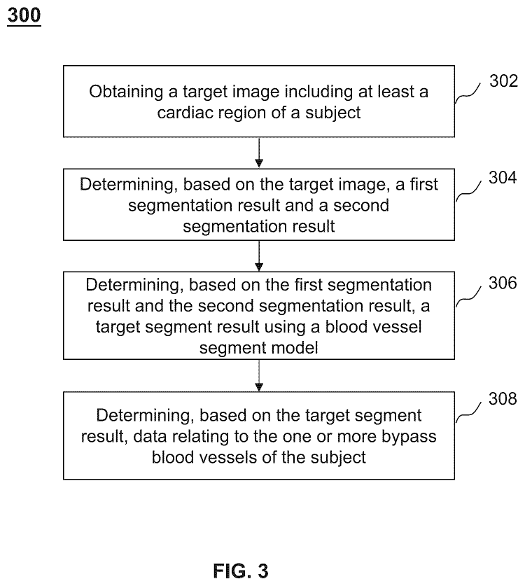

The acquisition module 202 may be configured to obtain information relating to the imaging system 100. For example, the acquisition module 202 may be configured to obtain a target image including at least a cardiac region of a subject. As used herein, the cardiac region of the subject refers to a region including the heart of the subject. The subject may include a biological subject and/or a non-biological subject that includes at least the cardiac region (or a portion thereof). More descriptions regarding the obtaining of the target image may be found elsewhere in the present disclosure. See, e.g., operation 302 in FIG. 3, and relevant descriptions thereof.

The determination module 204 may be configured to determine a first segmentation result and a second segmentation result based on the target image. The first segmentation result may indicate the heart of the subject segmented from the target image, and the second segmentation result may indicate vessels of the subject segmented from the target image. More descriptions regarding the determination of the first segmentation result and the second segmentation result may be found elsewhere in the present disclosure. See, e.g., operation 304 in FIG. 3, and relevant descriptions thereof.

The determination module may be also configured to determine a target segment result using a vessel segment model based on the first segmentation result and the second segmentation result. The target segment result may include a plurality of segment labels of a plurality of points on the vessels of the subject, and the plurality of segment labels may include a segment label corresponding to bypass vessels. More descriptions regarding the determination of the target segment result may be found elsewhere in the present disclosure. See, e.g., operation 306 in FIG. 3, and relevant descriptions thereof.

The determination module may be further configured to determine data relating to one or more bypass vessels of the subject based on the target segment result. In some embodiments, the data relating to the one or more bypass vessels may include first data relating to a starting point of each bypass vessel, second data relating to a path of each bypass vessel, third data relating to an anastomotic stoma between each bypass vessel and other vessels, or the like, or any combination thereof. More descriptions regarding the determination of the data relating to one or more bypass vessels of the subject may be found elsewhere in the present disclosure. See, e.g., operation 308 in FIG. 3, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, any one of the modules may be divided into two or more units. For instance, the acquisition module 202 may be divided into two units configured to acquire different data. In some embodiments, the processing device 140 may include one or more additional modules, such as a storage module (not shown) for storing data.

FIG. 3 is a flowchart illustrating an exemplary process 300 for determining data relating to one or more bypass vessels of a subject according to some embodiments of the present disclosure.

In some embodiments, the one or more bypass vessels of the subject may be one or more bypass vessels introduced by a CABG performed on the subject.

In 302, the processing device 140 (e.g., the acquisition module 202) may obtain a target image including at least a cardiac region of a subject.

As used herein, the cardiac region of the subject refers to a region including the heart of the subject. The subject may include a biological subject and/or a non-biological subject that includes at least the cardiac region (or a portion thereof). For example, the subject may be a human being, an animal, or a portion thereof. As another example, the subject may be a phantom that simulates a human cardiac region. In some embodiments, the subject may be a patient (or a portion thereof), and the target image may include at least the cardiac region of the patient. The heart may include the left atrium, the right atrium, the left ventricle, the right ventricle, vessels that are located at the cardiac region. In some embodiments, the vessels of the subject may include coronary arteries. In some embodiments, the vessels of the subject may further include one or more bypass vessels.

In some embodiments, the target image may include a 2D image (e.g., a slice image), a 3D image, a 4D image (e.g., a series of 3D images over time), and/or any related image data (e.g., scan data, projection data), or the like. In some embodiments, the target image may include a medical image (e.g., in the form of a digital imaging communication in medicine (DICOM) image file) generated by a biomedical imaging technique as described elsewhere in this disclosure. For example, the target image may be a medical image obtained using a Coronary computed tomography angiography (CCTA) technique. As another example, the target image may include a DR image, an MR image, a PET image, a CT image, a PET-CT image, a PET-MR image, an ultrasound image, etc. In some embodiments, the target image may include a 3D enhanced CT image.

In some embodiments, the target image may be generated based on image data acquired using the imaging device 110 of the imaging system 100 or an external imaging device. For example, the imaging device 110, such as a CT device, an MRI device, an X-ray device, a PET device, or the like, may be directed to scan the subject or a portion of the subject (e.g., the cardiac region of the subject). The processing device 140 may generate the target image based on image data acquired by the imaging device 110. In some embodiments, the target image may be previously generated and stored in a storage device (e.g., the storage device 150, the storage 220, the storage 390, or an external source). The processing device 140 may retrieve the target image from the storage device.

In 304, the processing device 140 (e.g., the determination module 204) may determine, based on the target image, a first segmentation result and a second segmentation result.

In some embodiments, the first segmentation result may indicate the heart of the subject segmented from the target image. In some embodiments, the first segmentation result may indicate multiple portions of the heart of the subject segmented from the target image. For example, the first segmentation result may indicate the left atrium, the right atrium, the left ventricle, the right ventricle, the aorta, etc., of the heart.

In some embodiments, the first segmentation result may be represented as a first segmentation image of the heart generated based on the target image. For example, the first segmentation result may be represented as a first segmentation mask of the heart generated based on the target image. Merely by way of example, portions corresponding to the left atrium, the right atrium, the left ventricle, the right ventricle, the aorta, and the aortic arch of the heart may be identified in the target image, and the first segmentation mask may be generated based on the identified portions. In the first segmentation mask, pixels (or voxels) corresponding to the left atrium, the right atrium, the left ventricle, the right ventricle, the aorta, the aortic arch, and other part (also referred to as a heart background region) of the subject may be displayed with different labels.

In some embodiments, the heart and/or different portions of the heart (e.g., the left atrium, the right atrium, the left ventricle, the right ventricle, the aorta, and the aortic arch of the heart) may be segmented from the target image manually by a user (e.g., a doctor, an imaging specialist, a technician) by, for example, drawing bounding boxes on the target image displayed on a user interface. Alternatively, the target image may be segmented by the processing device 140 automatically according to an image analysis algorithm (e.g., an image segmentation algorithm). For example, the processing device 140 may perform image segmentation on the target image using an image segmentation algorithm. Exemplary image segmentation algorithm may include a thresholding segmentation algorithm, a compression-based algorithm, an edge detection algorithm, a machine learning-based segmentation algorithm, or the like, or any combination thereof.

In some embodiments, the heart and/or different portions of the heart (e.g., the left atrium, the right atrium, the left ventricle, the right ventricle, the aorta, and the aortic arch of the heart) may be segmented from the target image using a first segmentation model. The first segmentation model may be a trained model (e.g., a machine learning model) used for heart segmentation. Merely by way of example, the target image may be inputted into the first segmentation model, and the first segmentation model may output the first segmentation result and/or information (e.g., position information and/or contour information) relating to the heart or different portions of the heart. In some embodiments, the first segmentation model may include a deep learning model, such as a deep neural network (DNN) model, a convolutional Neural Network (CNN) model, a recurrent neural network (RNN) model, a feature pyramid network (FPN) model, a generative adversarial network (GAN) model, or the like, or any combination thereof.

In some embodiments, the processing device 140 may obtain the first segmentation model from one or more components of the imaging system 100 (e.g., the storage device 150, the terminals(s) 130) or an external source via a network (e.g., the network 120). For example, the first segmentation model may be previously trained by a computing device (e.g., the processing device 140 or a computing device of a vendor of the first segmentation model), and stored in a storage device (e.g., the storage device 150, the storage 220, and/or the storage 390) of the imaging system 100. The processing device 140 may access the storage device and retrieve the first segmentation model. In some embodiments, the first segmentation model may be generated according to a machine learning algorithm. Merely by way of example, the first segmentation model may be trained according to a supervised learning algorithm using sample cardiac images and ground truth segmentation result of the heart and/or different portions of the heart in each sample cardiac image.

The second segmentation result may indicate vessels of the subject segmented from the target image. For example, the second segmentation result may indicate coronary arteries, the one or more bypass vessels, etc., of the subject.

In some embodiments, the second segmentation result may be represented as a second segmentation image (e.g., a second segmentation mask) of the vessels generated based on the target image. For example, the second segmentation result may be represented as a binary segmentation mask of the vessels generated based on the target image. A portion corresponding to the vessels of the subject may be identified in the target image, and the second segmentation mask may be generated based on the identified portion. As another example, the binary segmentation mask may be represented as a matrix in which elements having a label of “1” represent physical points of the vessels and elements having a label of “0” represent physical points of the vessel background region.

As another example, portions corresponding to different vessels of the subject may be identified in the target image, and the second segmentation mask may be generated based on the identified portions. In the second segmentation mask, pixels (or voxels) corresponding to different vessels (e.g., coronary arteries, the one or more bypass vessels, etc.), and the vessel background region of the subject may be displayed with different labels.

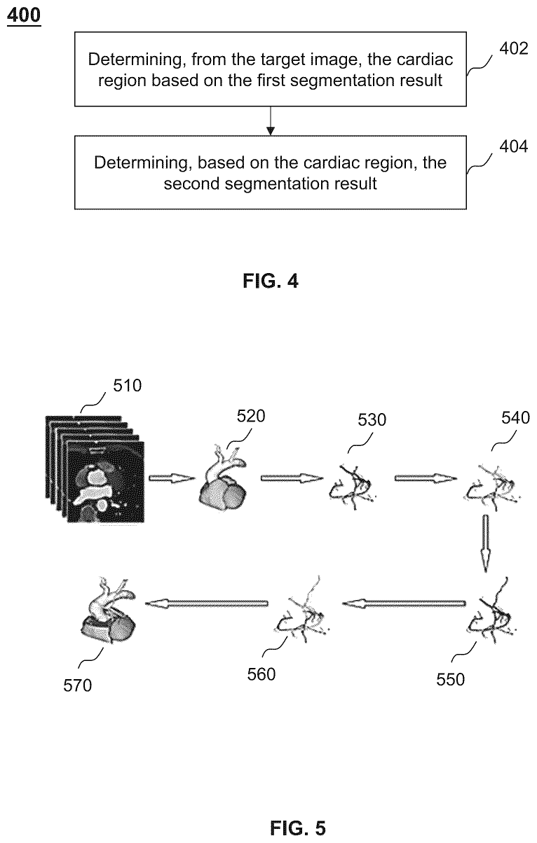

In some embodiments, the processing device 140 may determine a cardiac region from the target image based on the first segmentation result. The processing device 140 may further determine the second segmentation result based on the cardiac region. More descriptions regarding the determination of the second segmentation result based on the cardiac region may be found elsewhere in the present disclosure (e.g., FIG. 4 and the descriptions thereof).

In 306, the processing device 140 (e.g., the determination module 204) may determine, based on the first segmentation result and the second segmentation result, a target segment result using a vessel segment model (also referred to as a vessel parsing model).

In some embodiments, the target segment result may include a plurality of segment labels of a plurality of points on the vessels of the subject. As used herein, a segment label of a point may indicate which vessel segment the point is located at. For example, the plurality of segment labels may include a segment label corresponding to bypass vessels. For brevity, the segment label corresponding to bypass vessels may be referred to a bypass segment label. If a point has a bypass segment label, the point may be deemed as being located at a bypass vessel.

In some embodiments, the plurality of segment labels may include segment labels corresponding to a right coronary artery (RCA), a right posterior descending artery (R-PDA), a right posterior left ventricular branch (R-PLB), a left main coronary artery (LM), a left anterior descending artery (LAD), a diagonal branch (D), a left circumflex branch (LCX), an obtuse marginal ramus (OM), a left posterior descending artery (L-PDA), a left posterior left ventricular branch (L-PLB), an intermediate branch (RAMUS), a left atrial branch, an acute marginal branch (AM), a septal branch(S), a sinus nodal branch (LSN). In some embodiments, the target segment result may further include a background label corresponding to a background region other than the vessels of the subject in the target image.

The blood vessel segment model may be a trained model (e.g., a machine learning model) used for dividing vessels into vessel segments. Merely by way of example, the first segmentation result and the second segmentation result may be input into the vessel segment model, and the vessel segment model may output the target segment result. In some embodiments, the vessel segment model may include a deep learning model, such as a DNN model, a CNN model, a RNN model, a FPN model, a GAN model, or the like, or any combination thereof.

In some embodiments, the processing device 140 may determine a first distance map including distance information from each point to a ventricle region in the first segmentation result based on the first segmentation result. The ventricle region refers to a region corresponding to the left ventricle region and the right ventricle region of the subject in the first segmentation result. In some embodiments, the processing device 140 may generate a ventricle region mask based on the first segmentation result. For example, the ventricle region mask may be represented as a binary segmentation mask. In the ventricle region mask, the portion corresponding to the ventricle region in the first segmentation result may have a label of “1”, and the remaining portion (also referred to as a ventricle background region) of the first segmentation result may have a label of “0”. The processing device 140 may determine a minimum distance between each point in ventricle background region and the ventricle region. Further, the processing device may generate the first distance map based on the minimum distances between all points in ventricle background region and the ventricle region. For example, the first distance map may be represented as an image having the same size as the ventricle region mask, and in the first distance map, the value of each point corresponding to the ventricle region may be equal to 0, and the value of each point corresponding to the ventricle background region may be equal to its minimum distance to the ventricle region.

The processing device 140 may determine a second distance map including distance information from each point to an aorta region in the first segmentation result based on the first segmentation result. The aorta region in the first segmentation result refers to a region corresponding to the aorta and the aortic arch of the subject in the first segmentation result. In some embodiments, the processing device 140 may generate an aorta region mask based on the first segmentation result. For example, the aorta region mask may be represented as a binary segmentation mask. In the aorta region mask, the portion corresponding to the aorta region in the first segmentation result may have a label of “2”, and the remaining portion (also referred to as an aorta background region) of the first segmentation result may have a label of “3”. The processing device 140 may determine a minimum distance between each point in aorta background region and the aorta region. Further, the processing device 140 may generate the second distance map based on the minimum distances between all points in aorta background region and the aorta region. In some embodiments, the minimum distance between each point in ventricle background region and the ventricle region or the minimum distance between each point in aorta background region and the aorta region may include a Euclidean distance, a Manhattan distance, a cosine distance, an Minkowski distance, a Chebyshev distance, or the like.

Further, the processing device 140 may determine the target segment result using the vessel segment model based on the first segmentation result, the second segmentation result, the first distance map, and the second distance map. Merely by way of example, the first segmentation result, the second segmentation result, the first distance map, and the second distance map may be input into the vessel segment model, and the vessel segment model may output the target segment result. The vessels in the cardiac region and the one or more bypass vessels are usually located in or near the ventricle region and the aortic region. In addition, different vessel segments normally are located at different positions relative to the ventricle region and the aortic region. The first distance map and the second distance map may provide additional reference information for facilitating the vessel segment division, therefore the target segment result based on the first distance map and the second distance map may have an improved accuracy.

In some embodiments, the processing device 140 may combine the first segmentation result and the second segmentation result into a first combination segmentation result. The processing device 140 may determine the target segment result using the vessel segment model based on the first combination segmentation result, the first distance map, and the second distance map. For example, first combination segmentation result, the first distance map, and the second distance map may be input into the vessel segment model via three input channels, and the vessel segment model may output the target segment result via 17 output channels. The 17 output channels may include 16 channels corresponding to 16 segment labels and 1 channel corresponding to a background label as aforementioned.

In some embodiments, the vessel segment model may include a bypass vessel segment model. The target segment result may be determined using the bypass vessel segment model. The bypass vessel segment model may be capable of accurately identifying bypass vessels. In some embodiments, the obtaining of the bypass vessel segment model may be performed in a similar manner as that of the first segmentation model described in connection with operation 304.

In some embodiments, the bypass vessel segment model may be generated according to a machine learning algorithm. Merely by way of example, the bypass vessel segment model may be generated by training a first preliminary model using a plurality of first training samples. Each first training sample may include a sample first segmentation result, a sample second segmentation result, a sample first distance map, a sample second distance map, and a ground truth segment result of a sample subject. In some embodiments, the sample first segmentation result, the sample second segmentation result, the sample first distance map, the sample second distance map may be similar to the first segmentation result, the second segmentation result, the first distance map, the second distance map, respectively. In some embodiments, the sample first segmentation result and the sample second segmentation result may be sample images with a size in a range from 64*64*64 to 256*256*256. For example, the sample first segmentation result and the sample second segmentation result may be sample images with a size of 128*128*128. In some embodiments, one or more of the sample first segmentation result, the sample second segmentation result, the sample first distance map, the sample second distance map, or the ground truth segment result may be sample images with a resolution in a range from 0.6 mm to 2.0 mm. For example, the sample first segmentation result and the sample second segmentation result may be sample images with a resolution of 1.2 mm in a 3D space.

In some embodiments, the training of the first preliminary model may be performed according to a first loss function. For example, in each iteration in the training of the first preliminary model, the sample first segmentation result, a sample second segmentation result, the sample first distance map, the sample second distance map of each first training sample may input into the first preliminary model, and the first preliminary model may output a predicted segment result. The first loss function may be used to measure a discrepancy between the predicted segment result predicted by the first preliminary model in an iteration and the ground truth segment result. The training of the first preliminary model may be terminated if a termination condition is satisfied (e.g., that the value of the first loss function obtained in the certain iteration is less than a threshold value, that a certain count of iterations has been performed, that the first loss function converges such that the difference of the values of the first loss function obtained in a previous iteration and the current iteration is within a threshold value). In some embodiments, the first loss function may include a focal loss function, a log loss function, a cross-entropy loss function, a Dice loss function, or the like. In some embodiments, the first preliminary model may be trained using optimization algorithms such as an adaptive moment estimation (Adam) optimization algorithm, a stochastic gradient descent optimization algorithm, an AdamW optimization algorithm, a root mean square propagation (RMSprop) optimization algorithm, etc.

In some embodiments, a proportion of sample data relating to bypass vessels in the first training samples may be relatively high (e.g., higher than a threshold proportion), so that the obtained bypass vessel segment model can accurately identify bypass vessels. For example, the proportion of sample data relating to bypass vessels in the first training samples may be higher than that in second training samples for generating a normal vessel segment model, which will be described in FIG. 4.

In 308, the processing device 140 (e.g., the determination module 204) may determine, based on the target segment result, data relating to the one or more bypass vessels of the subject.

In some embodiments, the data relating to the one or more bypass vessels may include first data relating to a starting point of each bypass vessel, second data relating to a path of each bypass vessel, third data relating to an anastomotic stoma between each bypass vessel and other vessels, or the like, or any combination thereof. As used herein, an anastomotic stoma between a bypass vessel and other vessels refers to a connection point between the bypass vessel and other vessels. The first data relating to a starting point of each bypass vessel may include a position of the starting point of each bypass vessel. The second data relating to the path of each bypass vessel may include a length of the path, a direction of the path, positions of points on the path, etc. The third data relating to an anastomotic stoma between each bypass vessel and coronary arteries may include a position of the anastomotic stoma, etc.

In some embodiments, the processing device 140 may determine the data relating to the one or more bypass vessels of the subject by performing one or more operations on the target segment result. The one or more operations may include a transformation operation, a comparison operation, an arithmetic operation, a screening operation, an analysis operation, etc.

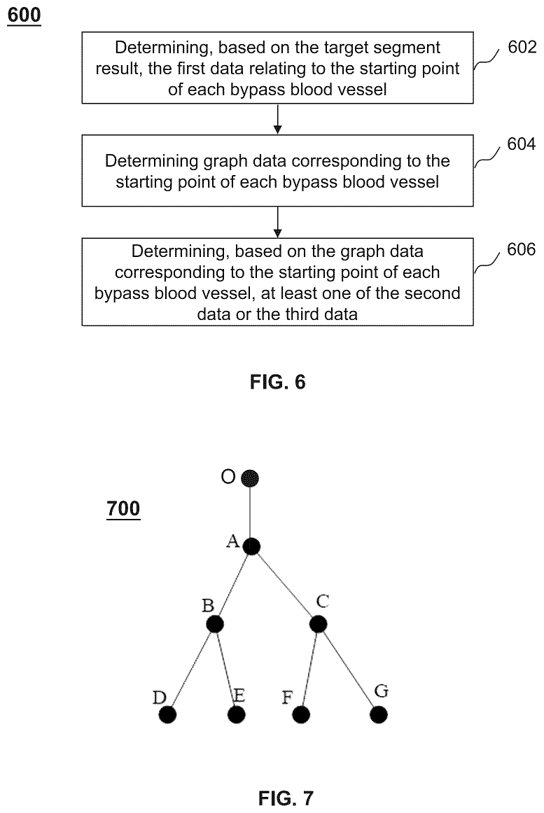

In some embodiments, the processing device 140 may determine the first data relating to the starting point of each bypass vessel based on the target segment result. Then, the processing device 140 may determine graph data corresponding to the starting point of each bypass vessel. In some embodiments, the graph data may be represented by a centerline tree that is generated based on points on one or more centerlines of the plurality of vessels and has the starting point as a root node. Further, the processing device 140 may determine the second data or the third data based on the graph data corresponding to the starting point of each bypass vessel. More descriptions regarding the determination of the first data, the second data, and the third data of each bypass vessel may be found elsewhere in the present disclosure (e.g., FIG. 6 and the descriptions thereof).

In some embodiments, after the data relating to the one or more bypass vessels of the subject is obtained, the processing device 140 may reconstruct an image (e.g., a 3D model image) of the one or more bypass vessels based on the data relating to the one or more bypass vessels using image reconstruction algorithms. In some embodiments, the reconstructed image of the one or more bypass vessels may also display other vessel segments and/or the heart of the subject. Merely by way of example, the processing device 140 may extract centerline information of at least a portion of the vessel segments, and then process the centerline information of at least the portion of vessel segments and the data relating to the one or more bypass vessels to obtain the reconstructed image of the one or more bypass vessels. Optionally, the reconstructed image may be sent to a terminal (e.g., the terminals 130) for a user (e.g., a doctor performing the CABG of the subject) to view, so that the user may evaluate a result of the CABG of the subject and formulate subsequent treatment plans of the subject.

As described elsewhere in the present disclosure, the conventional approach for determining the data relating to the bypass vessel involves a lot of human intervention since the bypass vessel cannot be divided into vessel segment from the image by the existing coronary segmentation and segment system, which is usually inefficient and/or susceptible to human errors or subjectivity. According to some embodiments of the present disclosure, the bypass vessel(s) may be automatically and accurately identified by the bypass vessel segment model. Further, the data relating to the bypass vessel(s) may be determined based on the target segment result with reduced or minimal or without user intervention. Compared with the conventional approach, the systems and methods of the present disclosure is more efficient and accurate by, e.g., reducing the workload of a user, cross-user variations, and the time needed for determining the data relating to the bypass vessel, thereby improving the accuracy and efficiency of the reconstruction of the bypass vessel(s).

FIG. 4 is a flowchart illustrating an exemplary process 400 for determining a second segmentation result according to some embodiments of the present disclosure. In some embodiments, one or more operations of the process 400 may be performed to achieve at least part of operation 304 as described in connection with FIG. 3.

In 402, the processing device 140 (e.g., the determination module 204) may determine, from the target image, a first region of the cardiac region based on the first segmentation result.

As described elsewhere in the present disclosure, the first segmentation result may indicate the heart of the subject segmented from the target image. In some embodiments, the heart of the subject may include a portion that includes coronary vessels. The processing device 140 may determine a region corresponding to the portion that includes coronary vessels from the target image based on the first segmentation result, and designate the region corresponding to the portion that includes coronary vessels as the first region of cardiac region.

In 404, the processing device 140 (e.g., the determination module 204) may determine, based on the first region of the cardiac region, the second segmentation result.

In some embodiments, the processing device 140 may generate the second segmentation result by segmenting vessels from the first region. In some embodiments, the vessels may be segmented from the first region in a similar manner as how the heart and/or different portions of the heart are segmented from the target image as described in connection with operation 304. For example, the vessels may be segmented from the first region manually or by the processing device 140 automatically. In some embodiments, vessels of the subject may be segmented from the first region using a second segmentation model. The second segmentation model may be a trained model (e.g., a machine learning model) used for vessel segmentation. Merely by way of example, the first region may be inputted into the second segmentation model, and the second segmentation model may output the second segmentation result and/or information (e.g., position information and/or contour information) relating to the vessels. In some embodiments, the second segmentation model may include a deep learning model, such as a DNN model, a CNN model, a RNN model, a FPN model, a GAN model, or the like, or any combination thereof. In some embodiments, the obtaining of the second segmentation model may be performed in a similar manner as that of the first segmentation model described in connection with operation 304.

In some embodiments, considering that there may be vessels located in the upper part of the heart of the subject, the processing device 140 may determine a second region from the target image based on the cardiac region, and determine the second segmentation result by segmenting vessels from the first region and the second region. The second region may be a region of the cardiac region other than the first region.

In some embodiments, the vessels in the second region and the vessels in the first region may be segmented jointly or separately. For example, the second region and the first region may be input into the second segmentation model together, and the second segmentation model may output the vessel segmentation result in both the second region and the first region as the second segmentation result. In some embodiments, the processing device 140 may generate an first vessel segmentation result by segmenting vessels from the first region. For example, the first region may be inputted into the second segmentation model, and the second segmentation model may output the first vessel segmentation result. Further, the processing device 140 may generate a second vessel segmentation result by segmenting vessels from the second region. In some embodiments, the determination of second vessel segmentation result may be performed in a similar manner as that of the first vessel segmentation result. For example, the second region may be inputted into the second segmentation model, and the second segmentation model may output the second vessel segmentation result. The processing device 140 may designate the combination of the second vessel segmentation result and the first vessel segmentation result as the second segmentation result.

In some cases, it is possible that there is no bypass vessel in the target image. During a diagnosis or treatment of heart disease of a patient, it may be necessary to determine whether there are one or more bypass vessels near the heart of the patient. If it is determined that there are one or more bypass vessels near the heart of the patient, data relating to the one or more bypass vessels of the patient needs to be determined for a comprehensive assessment of the heart disease. As another example, even if the target image is obtained after the CABG has been performed on the subject, it is possible that there is no bypass vessel in the target image for some reasons (e.g., due to imaging errors). Therefore, in some embodiments, before operation 404 is performed, the processing device 140 may determine whether there are one or more bypass vessels in the subject based on the first vessel segmentation result. In response to determining that there are one or more bypass vessels in the subject, the processing device 140 may continue to determine the second region from the target image, and generate the second segmentation result based on the second region and the first vessel segmentation result (or the cardiac region). In response to determining that there is no bypass vessel in the subject, the processing device 140 may obtain an additional image of the subject, and repeat process 300 and process 400 to determine data relating to one or more bypass vessels of the subject based on the additional image. In this way, only a small amount of computing resources is needed to firstly determine whether there are one or more bypass vessels in the subject based on the first vessel segmentation result, and the subsequent process may be continued only when it is determined that there are one or more bypass vessels in the subject, which may greatly save computing resources and improve efficiency of the determining data relating to one or more bypass vessels of a subject.

In some embodiments, to determine whether there are one or more bypass vessels in the subject, the processing device 140 may determine an initial segment result using the vessel segment model based on the first segmentation result and the first vessel segmentation result. The initial segment result may include a plurality of initial segment labels of a plurality of points on the vessels of the subject. In some embodiments, the first segmentation result and the first vessel segmentation result may be combined into a second combination segmentation result, and the processing device 140 may determine the initial segment result based on the second combination segmentation result. For example, the first segmentation result and the first vessel segmentation result (or the second combination segmentation result) may be input into the vessel segment model, and the vessel segment model may output the initial segment result. In some embodiments, the processing device 140 may obtain the first distance map and the second distance map as described in connection with operation 306. The first distance map, the second distance map, the first segmentation result, and the first vessel segmentation result may be input into the vessel segment model, and the vessel segment model may output the initial segment result.

Further, the processing device 140 may determine whether there are one or more bypass vessels in the subject based on the initial segment result. For example, the processing device 140 may determine an amount of points with the bypass segment label based on the initial segment result. The processing device 140 may determine whether the amount of points with the bypass segment label exceeds a segment amount threshold. In response to determine that the amount of points with the bypass segment label exceeds the segment amount threshold, the processing device 140 may determine that there are one or more bypass vessels in the subject. In some embodiments, the processing device 140 may determine a plurality of vessel connected components (also referred to as connected domains) based on the first vessel segmentation result. As used herein, a vessel connected component refers to a region formed by a certain number of neighboring vessel points in the first vessel segmentation result. In some embodiments, the plurality of vessel connected components may be determined by traversing neighbourhoods (e.g., 6-neighbourhoods, 8-neighbourhoods, or 26-neighbourhoods) of a plurality of points on the vessels of the cardiac region. Merely by way of example, the processing device 140 may determine one or more base points on the vessels of the cardiac region according to the first vessel segmentation result. For each base point, neighboring points that are on the vessels around the base point may be obtained by searching a 26-neighbourhoods of the base point; for each neighboring point, new neighboring points that are on the vessels around the neighboring point may be obtained by searching a 26-neighbourhoods of the neighboring point; repeating the above steps, each time a new neighboring point is obtained, and neighboring points that are on the vessels around the new neighboring point may be obtained by searching a 26-neighbourhoods of the new neighboring point until no new neighboring point are found. The processing device 140 may designate a region formed by the base point and the multiple neighboring points as a vessel connected component corresponding to the base point.

For each of the plurality of vessel connected components, the processing device 140 may determine feature information of the vessel connected component. In some embodiments, the feature information of the vessel connected component may include a distance from the vessel connected component to a ventricle region, a distance from the vessel connected component to an aorta region, a size of the vessel connected component, or the like, or any combination thereof. As used herein, a size of a vessel connected component refers to an amount of points in the vessel connected component. The processing device 140 may determine the size of the vessel connected component based on the points in the vessel connected component. The ventricle region refers to a region including the left ventricle region and the right ventricle region, and the aorta region refers to a region including the aorta and the aortic arch. The distance from the vessel connected component to the ventricle region may include, for example, a distance from a central point in the vessel connected component to the ventricle region, a shortest distance from the points in the vessel connected component to the ventricle region, a largest distance from the points in the vessel connected component to the ventricle region, or the like, or any combination thereof. The distance from the vessel connected component to the aorta region may include for example, a distance from a central point in the vessel connected component to the aorta region, a shortest distance from the points in the vessel connected component to the aorta region, a largest distance from the points in the vessel connected component to the aorta region, or the like, or any combination thereof. As described in connection with operation 306, the processing device 140 may determine the first distance map including distance information from each point to the ventricle region in the first segmentation result and the second distance map including distance information from each point to an aorta region in the first segmentation result. The processing device 140 may obtain, from the first distance map and the second distance map, the distance from the vessel connected component to the ventricle region and the distance from the vessel connected component to the aorta region, respectively.

The processing device 140 may determine whether there are one or more bypass vessels in the subject based on the feature information relating to the plurality of vessel connected components. Merely by way of example, for each vessel connected component, the processing device 140 may determine whether the distance from the vessel connected component to the ventricle region is greater than a first distance threshold, the distance from the vessel connected component to the aorta region is greater than a second distance threshold, and a size of the vessel connected component is smaller than or equal to a size threshold. In response to determining that the distance from the vessel connected component to the ventricle region is not greater than the first distance threshold, the distance from the vessel connected component to the aorta region is not greater than the second distance threshold, and the size of the vessel connected component is not smaller than the size threshold, the processing device 140 may determine whether an amount of points with the bypass segment label in the vessel connected component is greater than a first amount threshold. In response to determining that the amount of points with the bypass segment label is greater than the first amount threshold, the processing device 140 may determine that there are points on the bypass vessels in the vessel connected component, that is, the processing device 140 may determine that there are one or more bypass vessels in the subject. The first distance threshold, the second distance threshold, the size threshold, and the first amount threshold may be set according to an actual need.

In some embodiments, the vessel segment model may include a normal vessel segment model, and the initial segment result may be determined using the normal vessel segment model. The normal vessel segment model may be capable of accurately identifying vessels (e.g., coronary arteries and their branches) that are located at the cardiac region. The initial segment result may be determined using the normal vessel segment model. In some embodiments, the normal vessel segment model and the bypass vessel segment model described in FIG. 3 may have a same structure, but different model parameters.

In some embodiments, the normal vessel segment model may be generated by training a second preliminary model using second training samples in a similar manner as the generation of the bypass vessel segment model. In some embodiments, a proportion of sample data relating to bypass vessels in the second training samples may be relatively small, so that the obtained normal vessel segment model can accurately identify the vessels that located at the cardiac region. For example, the proportion of sample data relating to bypass vessels in the second training samples may be smaller than that in the second training samples.

FIG. 5 is a schematic diagram illustrating an exemplary process 500 for reconstructing one or more bypass vessels of a subject according to some embodiments of the present disclosure. As shown in FIG. 5, one or more images 510 (e.g., 3D images) may be obtained. Each of the one or more images 510 may include at least a cardiac region of a subject. A target image 510 may be obtained from the one or more images 510. A first segmentation result 520 may be determined by segmenting the heart from the target image 510, and then an first vessel segmentation result 530 may be determined based on the first segmentation result 520. Further, an initial segment result 540 may be determined based on the first segmentation result 520 and the first vessel segmentation result 530. If it is determined that there are one or more bypass vessels in the subject based on the initial segment result 540 and the first vessel segmentation result 530, a second segmentation result 550 may be determined based on the first vessel segmentation result 530 and a second vessel segmentation result, wherein the second vessel segmentation result may be obtained by segmenting vessels from a second region of the target subject. Then, a target segment result 560 may be determined based on the first segmentation result 520 and the second segmentation result 550. Finally, data relating to the one or more bypass vessels of the subject may be determined based on the target segment result, and a heart image 570 of the subject including the one or more bypass vessels may be reconstructed based on the data relating to the one or more bypass vessels.

FIG. 6 is a flowchart illustrating an exemplary process 600 for determining data relating to one or more bypass vessels according to some embodiments of the present disclosure. In some embodiments, one or more operations of the process 600 may be performed to achieve at least part of operation 308 as described in connection with FIG. 3.

As described elsewhere in the present disclosure, the data relating to the one or more bypass vessels may include first data relating to a starting point of each bypass vessel, second data relating to a path of each bypass vessel, third data relating to a anastomotic stoma between each bypass vessel and other vessels, or the like, or any combination thereof.

In 602, the processing device 140 (e.g., the determination module 204) may determine, based on the target segment result, the first data relating to the starting point of each bypass vessel.

As described in connection with operation 306, the target segment result may include a plurality of segment labels of a plurality of points on the vessels of the subject. In some embodiments, the target segment result may be represented as a target segment image including a plurality of vessel segments of the vessels.

In some embodiments, the processing device 140 may determine endpoints of centerlines of the vessels based on the target segment result. For example, the processing device 140 may obtain the centerlines of the plurality of vessel segments by performing a skeletonization processing on the target segment image. The processing device 140 may determine the endpoints of the centerlines based on points on the centerlines of the plurality of vessel segments. As used herein, endpoints of a centerline refer to points at both ends of the centerline. As another example, for each of the plurality of vessel segments, the processing device 140 may determine two vessel ending layers that located at both ends of the vessel segment. The processing device 140 may designate two centers of the vessel ending layers as the endpoints of the centerline of the vessel segment. Further, the processing device 140 may determine the centerline of the vessel segment using a maximum inscribed sphere algorithm based on the vessel segment and the endpoints of the centerline of the vessel segment.

The processing device 140 may determine one or more candidate starting points based on the target segment result from the endpoints of the centerlines of the plurality of vessels. Specifically, for each of the endpoints of the centerlines, the processing device 140 may determine a set of target points corresponding to the endpoint. For example, the processing device 140 may determine a preset number of points around the endpoint as the set of target points corresponding to the endpoint. The processing device 140 may determine whether a first condition that an amount of points with the bypass segment label in the set of target points is greater than a second amount threshold is satisfied, whether a second condition that a distance between the endpoint and the aorta of the subject is smaller than a third distance threshold is satisfied, and whether a third condition that a distance from the endpoint to a region above the ventricle and atrium region of the heart of the subject is greater than a fourth distance threshold is satisfied. The ventricle and atrium region of the heart may include the left atrium, the right atrium, the left ventricle, and the right ventricle, of the heart. The distance from the endpoint to the region above the ventricle and atrium region refers to a distance from the endpoint to the ventricle and atrium region along an axial direction (i.e., a direction from the head to the feet) of the subject. In response to determining that all of the first, second, and third condition is satisfied, the processing device 140 may determine the endpoint as a candidate starting point. Alternatively, in response to determining that at least one of the first, second, and third condition is satisfied, the processing device 140 may determine the endpoint as a candidate starting point. In some embodiments, the second amount threshold, the third distance threshold, and the fourth distance threshold may be set manually by a user (e.g., an engineer) according to an experience value or a default setting of the imaging system 100, or determined by the processing device 140 according to an actual need. For example, the second amount threshold may be a half of an amount of the target points, the third distance threshold may be 0.5 cm, and the fourth distance threshold may be 1.5 cm.

Further, the processing device 140 may determine the starting point of each bypass vessel based on the first segmentation result from the one or more candidate starting points. In some embodiments, one or more exclusion points may be determined from the one or more candidate starting points, and the processing device 140 may determine the remaining candidate starting point(s) other than the one or more exclusion points in the one or more candidate starting points as the one or more starting points of the one or more bypass vessels.

For example, the processing device 140 may determine a plurality of target vessel connected components based on the second segmentation result. In some embodiments, the plurality of target vessel connected components may be determined in a similar manner as how the plurality of vessel connected components are determined described in connection with operation 404. Further, the processing device 140 may determine whether there are at least two candidate starting points in a same target vessel connected component. In response to determining that there are at least two candidate starting points in a same target vessel connected component, the processing device 140 may determine a distance between each two of the at least two candidate starting points in a same target vessel connected component. If the distance between two candidate starting points in a same target vessel connected component is smaller than a fifth distance threshold (e.g., 0.5 cm), the processing device 140 may determine one of the candidate starting points further from the aorta as an exclusion point.

As another example, for a candidate starting point, the processing device 140 determine whether a minimum distance between the target vessel connected component where the candidate starting point is located and a surface of the ventricle and atrium region exceeds a sixth distance threshold (e.g., 0.5 cm). In response to determining that the minimum distance exceeds the sixth distance threshold, the processing device 140 may determine the candidate starting point as an exclusion point.

As yet another example, for a candidate starting point, the processing device 140 determine whether a distance between the candidate starting point and the surface of the ventricle and atrium region exceeds a seventh distance threshold (e.g., 5 cm). In response to determining that the distance between the candidate starting point and the surface of the ventricle and atrium region exceeds the seventh distance threshold, the processing device 140 may determine that the candidate starting point may be a point on a bypass vessel introduced by a left internal mammary artery bypass (LIMA) surgery or a right internal mammary artery bypass (RIMA) surgery. In this case, the processing device 140 determine whether an amount of points with the segment labels corresponding to the LAD and D in the target vessel connected component where the candidate starting point is located is smaller than a third amount threshold. In response to determining that the amount of points with the segment labels corresponding to the LAD and D is smaller than the third amount threshold, the processing device 140 may determine the candidate starting point as an exclusion point.

According to some embodiments of the present disclosure, the one or more candidate starting points may be determined from a small range (i.e., the endpoints of the centerlines of the plurality of vessels), which may improve the efficiency of the determination of the one or more candidate starting points, thereby improving the efficiency of the determination of one or more starting points of the one or more bypass vessels based on the one or more candidate starting points.

In 604, the processing device 140 (e.g., the determination module 204) may determine graph data corresponding to the starting point of each bypass vessel.

In some embodiments, the graph data corresponding to a starting point may be represented by a centerline tree that is generated based on points on one or more centerlines of the plurality of vessels and has the starting point as a root node. In some embodiments, the centerline tree may include nodes and edges. The nodes of the centerline tree may include a root node, one or more bifurcation points, and a plurality of termination points. Each edge of the centerline tree may connect two nodes of the centerline tree. For example, FIG. 7 is a schematic diagram illustrating an exemplary centerline tree 700 according to some embodiments of the present disclosure. As shown in FIG. 7, the centerline tree 700 may include a root node O, bifurcation points A, B, and C, termination points D, E, F, G, and edges OA, AB, AC, BD, BE, CF, and CG. Each of the edges OA, AB, AC, BD, BE, CF, and CG may connect two nodes of the centerline tree 700.

Merely by way of example, the processing device 140 may determine other points on the one or more centerlines in a neighborhood of the starting point (e.g., a 26-neighborhood). For each of the other points, the processing device 140 may determine a classification of the point. The classification of a point may include an edge point, a bifurcation point, and a termination point. In some embodiments, the processing device 140 may determine a classification of a point according to an amount of points on the one or more centerlines in a neighborhood of the point. If an amount of points on the one or more centerlines in the neighborhood of the point is greater than a fourth amount threshold (e.g., 2), the point may be a bifurcation point. If an amount of points on the one or more centerlines in the neighborhood of the point is equal to a first amount (e.g., 1), the point may be a termination point. If an amount of points on the one or more centerlines in the neighborhood of the point is equal to a second amount (e.g., 2), the point may be an edge point. If a point is a bifurcation point, a classification of each point on the one or more centerlines in a neighborhood of this bifurcation point may be determined. If a point is a termination point, the analysis for the neighborhood of this termination point does not need to be performed. In this way, all points on the one or more centerlines are traversed in turn. Then, starting from the starting point, the centerline tree may be generated by a connecting all the points in turn according to the classification of each point.

As another example, the processing device 140 may construct a spot map including points on the vessels of the subject based on the second segmentation result. Then, starting from the starting point, the centerline tree may be generated by connecting adjacent points in turn according to a position sequence of the points on the centerline in the spot map.

In 606, the processing device 140 (e.g., the determination module 204) may determine, based on the graph data corresponding to the starting point of each bypass vessel, at least one of the second data or the third data.

For illustration purposes, the determination of second data and third data of a target bypass vessel are described hereinafter. The second data of the target bypass vessel is referred to as target second data, and third data of the target bypass vessel is referred to as target third data.

In some embodiments, the processing device 140 may determine segment labels corresponding to the edges and the nodes of a target centerline tree corresponding to the target starting point of the target bypass vessel based on the target segment result. The processing device 140 may obtain segment labels corresponding to the nodes of the target centerline tree from the target segment result. For each edge of the target centerline tree, the processing device 140 may determine a vessel centerline segment that is located between the nodes connected by the edge. The processing device 140 may determine segment labels corresponding to all points on the vessel centerline segment (also referred to as edge points), and determine a segment label with a largest number as the segment label corresponding to the edge. That is, if most of the points on the vessel centerline segment has a target segment label, the processing device 140 may designate the target segment label as the segment label corresponding to the edge. For brevity, an edge with the bypass segment label may be also referred to as a bypass edge, an edge with one of the segment labels corresponding to the RCA, LCX, or LAD may be also referred to a trunk edge, and an edge with one of the segment labels other than the bypass segment label and the segment labels corresponding to the RCA, LCX, or LAD may be referred to as a branch edge.

Further, the processing device 140 may determine the target second data relating to a target path of the target bypass vessel based on the segment labels corresponding to the edges and the nodes of the target centerline tree. The processing device 140 may traverse nodes and edges of the target centerline tree from the root node (i.e., the target starting point) of the target centerline tree. Specifically, the processing device 140 may determine a bypass edge connected to the root node as a target edge. The processing device 140 may analyze other nodes of the target centerline tree other than the root node from a node connected to the target edge away from the root node in sequence. For each of the nodes of the target centerline tree other than the root node, if the node is a termination point or the ending of the RCA, LCX, or LAD, the processing device 140 may designate the node as a target termination point of the target path; if the node is a bifurcation point, the processing device 140 may analyze the bifurcation point. In particular, the processing device 140 may determine whether there are one or more bypass edges connected to the bifurcation point. In response to determining that there is only one bypass edge connected to the bifurcation point, the processing device 140 may directly select the bypass edge as a target edge connected with the bifurcation point.

In response to determining that there are a plurality of bypass edges connected to the bifurcation point, the processing device 140 may determine whether there are one or more bypass edge in the plurality of bypass edges belong one or more reference centerline trees. As used herein, a starting point of a bypass vessel other the target bypass vessel is referred to as a reference starting point, and a centerline tree corresponding to the reference starting point is referred to as a reference centerline tree. In other words, the processing device 140 may determine there are one or more bypass edges in the plurality of bypass edges connected to centerline trees of other starting points. For example, the processing device 140 determine whether there are one or more reference starting points on the target centerline tree. If there are no reference starting points on the target centerline tree, the processing device 140 may determine that there is no bypass edge in the plurality of bypass edges belong one or more reference centerline trees, that is, the processing device 140 may determine that the edges of the target centerline tree and one or more reference centerline trees intersect. If there are one or more reference starting points on the target centerline tree, for each of the one or more reference starting points, the processing device 140 may determine a shortest path connecting the reference starting point and the target starting point. The processing device 140 may determine whether an amount of edges on the shortest path that are bypass edges exceeds a fifth amount threshold. In response to determining that the amount of edges on the shortest path that are bypass edges exceeds the fifth amount threshold, the processing device 140 may determine that there are one or more bypass edges in the plurality of bypass edges belong one or more reference centerline trees, that is, the processing device 140 may determine that the edges of the target centerline tree and one or more reference centerline trees intersect. In this case, the processing device 140 may determine a plurality of angles between the plurality of bypass edges and a previous edge connected to the plurality of bypass edges. In some embodiments, for each of the plurality of bypass edges, the processing device 140 may determine included angles between vectors formed by connecting the bifurcation point and the other edge points on the bypass edge and a vector corresponding to the previous edge. The processing device 140 may determine an average of the included angles as the angle between the bypass edge and the previous edge. In some embodiments, for each of the plurality of bypass edges, the processing device 140 may determine another node connected to the bypass edge away from the bifurcation point. The processing device 140 may determine multiple points located on the vessel centerline segment corresponding to the bypass edge. For each of the bifurcation point and the multiple points, the processing device 140 may determine a vector from the another node to the point. Further, the processing device 140 may designate an average vector of the multiple vectors as a vector corresponding to the bypass edge. The process device 140 may determine a vector corresponding to the previous edge in a similar manner as how the vector corresponding to the bypass edge is determined. Then, the processing device 140 may determine an included angle between the vector corresponding to the previous edge and the vector corresponding to the bypass edge as the angle between the bypass edge and the previous edge. The processing device 140 may select the bypass edge with a minimum angle as a target edge.

In response to determining that the amount of edges on the shortest path that are bypass edges does not exceed the fifth amount threshold, the processing device 140 may determine that the edges of the target centerline tree and one or more reference centerline trees do not intersect. If it is determined that the edges of the target centerline tree and one or more reference centerline trees do not intersect, for each of the plurality of bypass edges, the processing device 140 may determine an amount of points with the bypass segment label on all edges connected to the bypass edge; if the amount of points with the bypass segment label on all edges connected to the bypass edge is greater than a sixth amount threshold, the processing device 140 may determine the bypass edge as a target edge connected with the bifurcation point of the target path.

In response to determining that there are no one or more bypass edges connected to the bifurcation point, the processing device 140 may determine whether there are one or more trunk edges connected to the bifurcation point. In response to determining that there are one or more trunk edges connected to the bifurcation point, the processing device 140 may determine the one or more trunk edges as one or more target edges of the target path. In response to determining that there are no one or more trunk edges connected to the bifurcation point, the processing device 140 may determine one or more target branch edges connected to the bifurcation point as one or more target edges connected with the bifurcation point of the target path. The one or more target branch edges may have a same segment label as a previous edge connected to the bifurcation point.

The processing device 140 may sequentially analyze all edges and nodes of the target centerline tree as described above. Finally, the processing device 140 may determine the target path by connecting the one or more target edges, the corresponding bifurcation points, and one or more target termination points in turn starting from the root node.

Merely by way of example, as shown in FIG. 7, the node O is the root node of the centerline tree 700, and the edge OA is a target edge connected to the root node O. If the edge AB connected to the node A is not a bypass edge, and the edge AC connected to the node A is a bypass edge, the edge AC may be determined a target edge. If the edge CF connected to the bifurcation point C is branch edge, and the edge CG connected to the bifurcation point C is trunk edge, the edge CG may be determined a target edge. The target path corresponding to the target starting point O may be the OACG.

In some embodiments, if the target path has a plurality of target termination points, the target path may have a plurality of target branches each of which corresponds a target anastomotic stoma between the target branch and other vessels. In some embodiments, for a target branch (that the target path have no branch may be deemed as one target branch), the processing device 140 may determine the target third data relating to the target anastomotic stoma between the target branch and other vessels by tracing the points on the target path from the target termination point of the target branch towards the target starting point of the target bypass vessel based on the segment label corresponding to the target termination point of the target branch. Specifically, if the segment label corresponding to the target termination point of the target branch is not the bypass segment label, the processing device 140 may trace the points on the target path until a point with the bypass segment label is tracked, and designate the point with the bypass segment label as the target anastomotic stoma between the target branch and other vessels. If the segment label corresponding to the target termination point of the target branch is the bypass segment label, the processing device 140 may trace the points on the target path until a point with other segment label except the bypass segment label is tracked within a tracking distance smaller than an eighth distance threshold (e.g., 3 cm), and designate the point with the other segment label other than the bypass segment label as the target anastomotic stoma between the target branch and other vessels. If the segment label corresponding to the target termination point of the target branch is the bypass segment label and a point with other segment label except the bypass segment label has not been tracked when the tracking distance exceeds the eighth distance threshold, the processing device 140 may designate the target termination point as the target anastomotic stoma between the target branch and other vessels.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof. For example, the operations of the illustrated processes 300, 400, and 600 are intended to be illustrative. In some embodiments, the processes 300, 400, and 600 may be accomplished with one or more additional operations not described, and/or without one or more of the operations discussed. Additionally, the order in which the operations of the processes 300, 400, and 600 and regarding descriptions are not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques.

Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

1. A system, comprising:
at least one storage device including a set of instructions for medical imaging; and
at least one processor in communication with the at least one storage device, wherein when executing the set of instructions, the at least one processor is configured to direct the system to perform operations including:
obtaining a target image including at least a cardiac region of a subject;
determining, based on the target image, a first segmentation result and a second segmentation result, wherein the first segmentation result indicates the heart of the subject segmented from the target image, and the second segmentation result indicates vessels of the subject segmented from the target image;
determining, based on the first segmentation result and the second segmentation result, a target segment result using a vessel segment model, wherein the target segment result includes a plurality of segment labels of a plurality of points on the vessels of the subject, and the plurality of segment labels include a segment label corresponding to bypass vessels; and
determining, based on the target segment result, data relating to one or more bypass vessels of the subject.

2. The system of claim 1, wherein the vessels of the subject include coronary arteries.

3. The system of claim 1, wherein the determining, based on the target image, a first segmentation result and a second segmentation result includes:
determining, from the target image, a first region of the cardiac region based on the first segmentation result; and
determining, based on the first region of cardiac region, the second segmentation result.

4. The system of claim 3, wherein the determining, based on first region of the cardiac region, the second segmentation result includes:
determining, from the target image, a second region of the cardiac region other than the first region; and
determining the second segmentation result by segmenting vessels from the first second region and the second region.

5. The system of claim 4, wherein the determining, from the target image, a second region of the cardiac region other than the first region includes:
generating a first vessel segmentation result by segmenting vessels from the first region;
determining, based on the first segmentation result and the first vessel segmentation result, an initial segment result using the vessel segment model;
determining, based on the initial segment result, whether there are one or more bypass vessels in the subject;
in response to determining that there are one or more bypass vessels in the subject, determining the second region from the target image.

6. The system of claim 5, wherein
the vessel segment model includes a normal vessel segment model and a bypass vessel segment model, wherein the bypass vessel segment model is trained using first training samples, the normal vessel segment model is trained using second training samples, and a proportion of sample data relating to bypass vessels in the first training samples is greater than that in the second training sample,
the initial segment result is determined using the normal vessel segment model, and
the target segment result is determined using the bypass vessel segment model.

7. The system of claim 5, wherein the determining, based on the initial segment result, whether there are one or more bypass vessels in the subject includes:
determining, based on the first vessel segmentation result, a plurality of vessel connected components;
for each of the plurality of vessel connected components, determining feature information of the vessel connected component, the feature information of the vessel connected component including at least one of a distance from the vessel connected component to a ventricle region, a distance from the vessel connected component to an aorta region, or a size of the vessel connected component; and
determining, based on the feature information relating to the plurality of vessel connected components, whether there are one or more bypass vessels in the subject.

8. The system of claim 1, wherein the determining, based on the first segmentation result and the second segmentation result, a target segment result using a vessel segment model includes:
determining, based on the first segmentation result, a first distance map including distance information from each point to a ventricle region in the first segmentation result;
determining, based on the first segmentation result, a second distance map including distance information from each point to an aorta region in the first segmentation result; and
determining the target segment result using the vessel segment model based on the first segmentation result, the second segmentation result, the first distance map, and the second distance map.

9. The system of claim 1, wherein the data relating to the one or more bypass vessels includes at least one of:
first data relating to a starting point of each bypass vessel,
second data relating to a path of each bypass vessel, or
third data relating to an anastomotic stoma between each bypass vessel and coronary arteries.

10. The system of claim 9, wherein the first data relating to the starting point of each bypass vessel is determined by:
determining, based on the target segment result, endpoints of centerlines of the plurality of vessels;
determining, from the endpoints of the centerlines of the vessels, one or more candidate starting points based on the target segment result; and
determining, from the one or more candidate starting points, the starting point of each bypass vessel based on the first segmentation result.

11. The system of claim 9, wherein the determining, based on the target segment result, data relating to one or more bypass vessels includes:
determining, based on the target segment result, the first data relating to the starting point of each bypass vessel;
determining graph data corresponding to the starting point of each bypass vessel, wherein the graph data is represented by a centerline tree that is generated based on points on one or more centerlines of the vessels and has the starting point as a root node;
determining, based on the graph data corresponding to the starting point of each bypass vessel, at least one of the second data or the third data.

12. The system of claim 11, wherein the centerline tree includes nodes and edges, and the determining, based on the graph data corresponding to the starting point of each bypass vessel, at least one of the second data or the third data includes:
determining, based on the target segment result, segment labels corresponding to the edges and the nodes of the centerline tree; and
determining, based on the segment labels corresponding to the edges of the centerline tree and the nodes of the centerline tree, the second data relating to the path of each bypass vessel.

13. The system of claim 11, wherein the determining, based on the graph data corresponding to the starting point of each bypass vessel, at least one of the second data or the third data includes:
for each of the one or more bypass vessel,
determining, based on the second data relating to the path of the bypass vessel, a termination point of the path of the bypass vessel and the segment label corresponding to the termination point of the path of the bypass vessel; and
determining the third data relating to the anastomotic stoma between the bypass vessel and other vessels by tracing the points on the path of the bypass vessel from the termination point towards the starting point of the bypass vessel based on the segment label corresponding to the termination point of the path of the each bypass vessel.

14. A method, the method being implemented on a computing device having at least one storage device and at least one processor, the method comprising:
obtaining a target image including at least a cardiac region of a subject;
determining, based on the target image, a first segmentation result and a second segmentation result, wherein the first segmentation result indicates the heart of the subject segmented from the target image, and the second segmentation result indicates vessels of the subject segmented from the target image;
determining, based on the first segmentation result and the second segmentation result, a target segment result using a vessel segment model, wherein the target segment result includes a plurality of segment labels of a plurality of points on the vessels of the subject, and the plurality of segment labels include a segment label corresponding to bypass vessels; and
determining, based on the target segment result, data relating to one or more bypass vessels of the subject.

15. The method of claim 14, wherein the vessels of the subject include coronary arteries.

16. The method of claim 14, wherein the determining, based on the target image, a first segmentation result and a second segmentation result includes:
determining, from the target image, a first region of the cardiac region based on the first segmentation result; and
determining, based on the first region of cardiac region, the second segmentation result.

17. The method of claim 16, wherein the determining, based on first region of the cardiac region, the second segmentation result includes:
determining, from the target image, a second region of the cardiac region other than the first region; and
determining the second segmentation result by segmenting vessels from the first second region and the second region.

18. The method of claim 17, wherein the determining, from the target image, a second region of the cardiac region other than the first region includes:
generating a first vessel segmentation result by segmenting vessels from the first region;
determining, based on the first segmentation result and the first vessel segmentation result, an initial segment result using the vessel segment model;
determining, based on the initial segment result, whether there are one or more bypass vessels in the subject;
in response to determining that there are one or more bypass vessels in the subject, determining the second region from the target image.

19. The method of claim 18, wherein
the vessel segment model includes a normal vessel segment model and a bypass vessel segment model, wherein the bypass vessel segment model is trained using first training samples, the normal vessel segment model is trained using second training samples, and a proportion of sample data relating to bypass vessels in the first training samples is greater than that in the second training sample,
the initial segment result is determined using the normal vessel segment model, and
the target segment result is determined using the bypass vessel segment model.

20-27. (canceled)

28. A non-transitory computer readable medium, comprising at least one set of instructions, wherein when executed by one or more processors of a computing device, the at least one set of instructions causes the computing device to perform a method, the method comprising:
obtaining a target image including at least a cardiac region of a subject;
determining, based on the target image, a first segmentation result and a second segmentation result, wherein the first segmentation result indicates the heart of the subject segmented from the target image, and the second segmentation result indicates vessels of the subject segmented from the target image;
determining, based on the first segmentation result and the second segmentation result, a target segment result using a vessel segment model, wherein the target segment result includes a plurality of segment labels of a plurality of points on the vessels of the subject, and the plurality of segment labels include a segment label corresponding to bypass vessels; and
determining, based on the target segment result, data relating to one or more bypass vessels of the subject.

29. (canceled)
